(19) United States
(12) Patent Application Publication
(10) Pub. No.: US 2022/0054475 A1
(43) Pub. Date: Feb. 24, 2022

(54) USING CATEQUENTINIB (ANLOTINIB) COMBINING WITH STANDARD CHEMOTHERAPY OR IMMUNOTHERAPY IN SEQUENTIAL ORDER FOR THE CANCER TREATMENT

(71) Applicant: ADVENCHEN PHARMACEUTICALS, LLC, Moorpark, CA (US)

(72) Inventors: Guoqing Paul Chen, Moorpark, CA (US); Judy Chen, Moorpark, CA (US); Zhe Li, Moorpark, CA (US); Yingyin Li, Moorpark, CA (US)

(21) Appl. No.: 17/465,492

(22) Filed: Sep. 2, 2021

Related U.S. Application Data
(63) Continuation of application No. PCT/US2020/021457, filed on Mar. 6, 2020.
(60) Provisional application No. 62/876,181, filed on Jul. 19, 2019.
(60) Provisional application No. 62/815,266, filed on Mar. 7, 2019.

(51) Int. Cl. A61K 31/4709; A61K 31/555; A61K 31/337; A61K 33/243; C07K 16/28; G16H 20/10; A61P 35/04

(52) U.S. Cl. CPC A61K 31/4709; A61K 31/555; A61K 31/337; A61P 35/04; C07K 16/2818; C07K 16/2827; G16H 20/10; A61K 33/243

(57) ABSTRACT

The present invention relates to a chemo combination therapy regimen to treat cancer. More specifically, the present invention relates to a novel chemo combination therapy regimen which relates to the combination of compound AL3818 (anlotinib, catequentinib) or its pharmaceutically acceptable salts with standard platinum-based and other chemotherapy agents or immunotherapy agents. The combination of these agents should be able to provide higher efficacy than employing any agent individually.

### CROSS REFERENCE TO RELATED APPLICATIONS

The present application is a continuation of International Patent Application No. PCT/US2020/021457, filed Mar. 6, 2020, which claims the benefit of priority to U.S. Provisional Patent Application No. 62/876,181, filed Jul. 19, 2019, and U.S. Provisional Patent Application No. 62/815,266, filed Mar. 7, 2019, the entirety of each of which is hereby incorporated by reference herein. Any and all applications for which a foreign or domestic priority claim is identified in the Application Data Sheet as filed with the present application are hereby incorporated by reference under 37 CFR 1.57.

### FIELD OF THE INVENTION

The present invention relates to a chemo combination therapy regimen to treat cancer. More specifically, the present invention relates to a novel chemo combination therapy regimen which relates to the combination of compound AL3818 (anlotinib, catequentinib) or its pharmaceutically acceptable salts with standard platinum-based and other chemotherapy agents or immunotherapy agents. The combination of these agents should be able to provide higher efficacy than employing any agent individually.

### BACKGROUND OF INVENTION

Currently, cancer is typically treated by one or a combination of methodologies, which include surgery, radiation therapy, chemotherapy and immunotherapy. In the past decades, rapid advances in chemotherapy were observed, which could provide much more possibility for reducing the mortality caused by cancer. Recent years, immunotherapy has gained enormous advancement in cancer therapy as well.

Paclitaxel is a well-known chemotherapy medication used to treat a number of types of cancer. The mechanism of action for paclitaxel is the paclitaxel could stabilize the microtubule polymer in the cells and protects them from disassembly. This function could block the mitosis process of cancer cells.

Carboplatin and cisplatin are both traditional platinum-based chemotherapeutic agents that used to treat various types of cancer. The mechanism of action for them is the platinum-based agents are able to form intra- or inter-linkage of DNA molecules in the cell. This manipulation can modify DNA structure and inhibits its synthesis, especially in cancer cells.

Protein tyrosine kinases (PTKs) are a series of enzymes that catalyze the phosphorylation of tyrosine residues in proteins. They play an important role in the cellular signal transduction cascades from extracellular signals through membrane to the cytoplasm and even nucleus. According to the different structures of the extracellular domains, they can be classified as epidermal growth factor receptor (EGFR), platelet-derived growth factor receptor (PDGFR), vascular endothelial growth factor receptor (VEGFR), fibroblast growth factor receptor (FGFR) and so on. Lots of studies have revealed that normal cells usually show no activity or low activity of PTKs, whereas many cancer cells feature over expression of PTKs. Obviously, the unusual hyperactivity of PTKs is closely correlated with the tumor cell growth and angiogenesis. For this reason, interrupting or blocking the activity of PTKs could dramatically suppress the growth of tumor cells. As a result, seeking for a PTK inhibitor with improved efficacy and safety profile has become an important target for designing and developing potential new anticancer drugs.

AL3818 (anlotinib, INN: catequentinib) is a novel multi-target receptor tyrosine kinase inhibitor. Study results have demonstrated that the compound AL3818 could inhibit the activities of tyrosine kinases such as vascular endothelial growth factor receptor (VEGFR1, VEGFR2/KDR, and VEGFR3). It has also demonstrated to be a strong inhibitor of fibroblast growth factor receptor (FGFR1, FGFR2, and FGFR3), platelet-derived growth factor receptor (PDGFR-α) and even the stem cell factor receptor (c-KIT) as well. This strong combination inhibition capability makes AL3818 (anlotinib) as a good candidate for multi-target kinase inhibitor that has the potential to express higher efficacy and less toxic than any other competitive products already been approved or still in development (see Shen et al. Journal of Hematology & Oncology 2018 11:120).

U.S. Pat. No. 8,148,532 disclosed the compound AL3818 and US patent 20190002435 disclosed a mouse model of combining chemotherapy agents with AL3818 to show synergistic anti-tumor effects.

The present invention describes a combination therapy regimen, which based on the sequential administration of standard platinum-based chemotherapy (or immunotherapy) and compound AL3818 or its pharmaceutical acceptable salts. This action is trying to use compound AL3818 or its pharmaceutical acceptable salts to inhibit the activities of protein tyrosine kinases (PTKs), thereby to inhibit the angiogenesis, which is closely related to the development, invasion, and metastasis of tumors.

### SUMMARY OF THE INVENTION

The present invention provides a regimen method to treat cancer in a subject in need thereof comprising: a standard platinum-based chemotherapeutic agent and another chemotherapeutic agent; or each individually; or an immunotherapeutic agent; in combing with AL3818 or its pharmaceutically acceptable salts in repeated cycles with the option of administration of maintenance mono therapy of AL3818 or its pharmaceutically acceptable salts.

More specifically, this present invention provides a chemo or immuno combination therapy regimen that used for treating cancer in human.

Accordingly, this chemo or immuno combination therapy regimen is administering to a subject in need thereof a chemotherapeutic or immunotherapy agent altogether with a kind of tyrosine kinase inhibitor.

In some embodiments, the chemotherapeutic or immunotherapy agent and tyrosine kinase inhibitor are administered cyclically in sequence.

The administration one cycle of the chemotherapeutic or immunotherapy agent can range from 2 to 6 weeks (eg. 3 weeks, 4 weeks, 5 weeks and 6 weeks). In the presented chemo combination therapy regimen, the chemotherapeutic or immunotherapy agent is administered periodically on a 3 weeks cycle (21-day cycle) at once a cycle, or 4 weeks (28-day cycle) at once or twice a cycle. In the presented combination therapy regimen, the chemotherapeutic or immunotherapy agent is preferably administered on a 3 weeks cycle (21-day cycle) at once per cycle.

The administration cycle of tyrosine kinase inhibitor can range from 1 to 4 weeks. In the presented chemo combination therapy regimen, the tyrosine kinase inhibitor is preferably administered periodically on a 3 weeks cycle (21-day cycle).

The described 21-day cycles include administration periods and therapy free periods. The chemotherapy or immunotherapy agent is administrated to the subject on the first day (Day 1) of the 21-day cycle. The tyrosine kinase inhibitor is administrated from Day 1-21 once or twice a day. The tyrosine kinase inhibitor is preferably administrated to the subject once daily for two weeks from Day 1 to Day 14 or Day 8 to Day 21 to have 7 days (Day 15 to Day 21 or Day 1 to Day 7) of drug free period. Therefore, this described administration regimen features an initial administration of the chemotherapeutic or immunotherapy agent or a combination of 2-3 agents, 2 weeks of tyrosine kinase inhibitor administration period and 7 days of tyrosine kinase inhibitor free period. The administration process repeats and follows the same regimen.

In some embodiments, the described periodic chemo combination therapy comprises at least 1-10 cycles of chemotherapy or immunotherapy agent(s), preferably 1-6 cycles of chemotherapy or immunotherapy agent(s). After the completion of 1-10 cycles of the chemo combination therapy, the tyrosine kinase inhibitor could be administrated individually without the utilization of chemotherapeutic nor immunotherapy agent(s) for maintenance therapy which can be used for 1-2 years, preferably 0.5-1 year.

In some embodiments, the described periodic chemo combination therapy comprises continuing treatment of chemotherapy or immunotherapy agent(s) with the tyrosine kinase inhibitor until patient intolerability or progression disease.

The tyrosine kinase inhibitor is AL3818 or its pharmaceutical acceptable salts, or its crystallines can be administrated, but not limited, at equal to or lower than 12 mg per day during chemo or immuno combination therapy period and equal to or lower than 16 mg per day during maintenance therapy period. Preferably 8 mg per day during chemo or immuno combination therapy period; and 8 mg, 10 mg or 12 mg per day during maintenance therapy period. In some special circumstances, the dosage strength of compound AL3818 or its pharmaceutical acceptable salts might be acceptable at 16 mg.

The dosage described at here was calculated according to the form of free base.

In a particular embodiment, the chemotherapeutic agents are the standard chemotherapy agents including gemcitabine, carboplatin, cisplatin, paclitaxel or carboplatin+paclitaxel and cisplatin+paclitaxel.

The described chemo or immuno combination therapy regimen can be applied for treating tumors including but not limited to ovarian cancer, endometrial cancer or cervical cancer.

In the embodiments of ovarian cancer or endometrial cancer, the chemotherapy agents are paclitaxel and carboplatin. In the embodiments of cervical cancer, the chemotherapy agents are paclitaxel and cisplatin. In the embodiments of platinum resistant ovarian cancer, the chemotherapy agent is selected from paclitaxel (weekly), pegylated liposomal doxorubicin (PLD) and topotecan.

In this described chemo combination therapy regimen, the tyrosine kinase inhibitor includes, but not limited to imatinib mesylate, sunitinib malate, erlotinib hydrochloride, dasatinib, lapatinib mesylate, nilotinib, gefitinib, and icotinib hydrochloride. In a particular embodiment, the tyrosine kinase inhibitor is compound AL3818 (anlotinib).

In some embodiments, the daily dosage of compound AL3818 is from 6 mg-16 mg which is calculated towards the content of free base. In more specific embodiments, the daily dosage of compound AL3818 is selected from 6 mg, 8 mg, 10 mg, 12 mg, 14 mg and 16 mg. The preferred daily dosage of compound AL3818 in the stage of chemo combination therapy is 8 mg. The preferred daily dosage of compound AL3818 in the stage of mono-therapy of maintenance period can be selected from 8 mg, 10 mg and 12 mg which follows two weeks on and one week off pattern. In some special scenarios, the compound AL3818 or its pharmaceutical acceptable salts might be bearable at a dosage of 16 mg.

The details for one or more embodiments of the invention are described in FIG. 1. Other necessary objects and features of this invention will be apparent from the description and from the claims.

### BRIEF DESCRIPTION OF THE DRAWINGS

FIG. 1 is a flow chart of a 21-Day Cycle of the Chemo Combination Therapy Regimen.

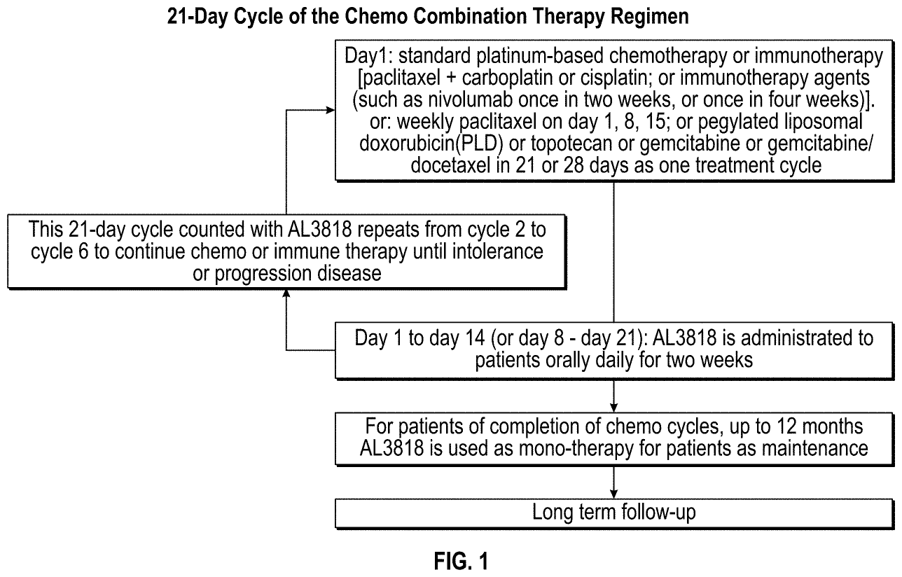

FIG. 2 is a chart displaying the efficacy of single Anlotinib Hydrochloride/control, and combined with Anti-PD-1 Antibody in the treatment of subcutaneously implanted hepatocellular carcinoma H22 in mice.

FIG. 3 is a graph displaying the influence of single Anlotinib Hydrochloride/Control and Combined with Anti-PD-1 Antibody on the growth of subcutaneously implanted hepatocellular carcinoma H22 in mice.

### DETAILED DESCRIPTION OF THE PREFERRED EMBODIMENT

### Compound AL3818

This present invention provides a therapy regimen for treating cancer, which relates to administrate a daily dosage of compound AL3818 (anlotinib, INN: catequentinib) to patients.

The chemical name of AL3818 (anlotinib) is (1-[[4-(4-fluoro-2-methyl-1H-indol-5-yloxy)-6-methoxyquinolin-7-yl]oxy]methyl]cyclopropaneamine, which features the following chemical structure:

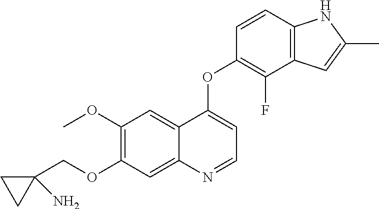 [SMILES: COc1cc2c(Oc3ccc4[nH]c(C)cc4c3F)ccnc2cc1OCC1(N)CC1]

The compound AL3818 (anlotinib) can be administrated to the patients in the form of free base. It can also be administrated in the form of salts, hydrates and prodrugs (will be converted into the free base form in vivo). In this described embodiments, AL3818 is administrated in the form of pharmaceutically acceptable salts.

The conception of “pharmaceutically acceptable salts” includes, but not limited to acid addition salts formed from inorganic acids such as hydrochloric acid, hydrobromic acid, sulfuric acid, nitric acid, phosphoric acid or the like; or acid addition salts formed from organic acids such as 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, isobutyric acid, lactic acid (DL), lactobionic acid, lauric acid, maleic acid, malic acid (-L), malonic acid, mandelic acid (DL), methanesulfonic acid, naphthalene-1,5-disulfonic acid, naphthalene-2-sulfonic acid, nicotinic acid, oleic acid, oxalic acid, palmitic acid, pamoic acid, phosphoric acid, proprionic acid, pyroglutamic acid (-L), salicylic acid, sebacic acid, stearic acid, succinic acid, tartaric acid (+L), thiocyanic acid, toluenesulfonic acid (p), undecylenic acid and the like (see P. H. Stahl and C. G. Wermuth, editors, Handbook of Pharmaceutical Salts: Properties, Selection and Use, Weinheim/Zurich:Wiley-VCH/VHCA, 2002).

A preferred pharmaceutically acceptable salt of compound AL3818 is the hydrochloride salt. In this described embodiment, compound AL3818 (anlotinib) is administrated in the form of dihydrochloride salt. Another preferred pharmaceutically acceptable salt of compound AL3818 is the maleic acid salt. In this described embodiment, compound AL3818 is administrated in the form of dimaleic acid salt.

Compound AL3818 (anlotinib) or its pharmaceutically acceptable salts can be administrated to patients via various administration routes, and these routes include, but not limited to: orally, parenterally, intraperitoneally, intravenously, intraarterially, transdermally, sublingually, intramuscularly, rectally, transbuccally, intranasally, via inhalation, vaginally, intraoccularly, via local administration, subcutaneously, intraadiposally, intraarticularly, intraperitoneally or intrathecally. In this described embodiment, the administration is performed orally.

The pharmaceutical compositions of compound AL3818 (anlotinib) or its pharmaceutically acceptable salts suitable for oral administration include, but not limited to tablets, capsules, dusts, granulates, drip pills, pastes, powders and tinctures. Tablets and capsules are the preferred pharmaceutical compositions among them. In a certain embodiment, capsules are the more preferred pharmaceutical compositions.

The present invention provides a therapy regimen for treating cancer, which comprises administrating a daily orally dosage of 6-16 mg compound AL3818 (anlotinib) or its pharmaceutically acceptable salts to patients. In an embodiment, compound AL3818 (anlotinib) is administrated in the form of pharmaceutically acceptable salts to the patient. In a further embodiment, compound AL3818 (anlotinib) is administrated in the form of dihydrochloride salt to patients. In yet a further embodiment, compound AL3818 (anlotinib) is administrated in the form of dihydrochloride salt in capsules to patients. The daily orally dosage of compound AL3818 dihydrochloride salt includes, but not limited to 6 mg, 8 mg, 10 mg, 12 mg, 14 mg and 16 mg which was calculated towards the content of free base. Another preferred pharmaceutically acceptable salt of compound AL3818 is the maleic acid salt. The dimaleic acid salt of compound AL3818 is administrated to the patient with or without pharmaceutical excipients. It is preferred with pharmaceutical excipients to be used as a capsule.

“Patients” refer to mammal, preferably human.

### 21-Day Cycle

To meet the existing need, the present invention provides a chemo combination therapy regimen for treating cancer, which comprises an interval and sequential administration of compound AL3818 (anlotinib) or its pharmaceutically acceptable salts in combination with standard platinum-based chemotherapy or immunotherapy to patients.

In the standard chemotherapy or immunotherapy, 21 days is preferably been selected as one treatment cycle. This presented chemo combination therapy regimen takes the advantage of the 21-day cycle and intervally administrates the compound AL3818 (anlotinib) or its pharmaceutically acceptable salts to patients in the rest period.

As a kind of novel multi-target receptor tyrosine kinase inhibitor, compound AL3818 or its pharmaceutically acceptable salts could suppress the abnormal hyperactivity of protein tyrosine kinases (PTKs) in cancer cells. This action could inhibit the angiogenesis process of metastatic cancers, thus inhibit the development, invasion, and metastasis of tumors in the therapy free period of the standard chemotherapy or immunotherapy.

### Example 1: AL3818 Dihydrochloride Phase 1 Chemo Combination Dosing De-Escalation Trial in Gynecologic Oncology Patients

A Phase 1 trial was designed to determine the recommended phase 2 dose (RP2D) for part 2 (phase 2a) of the study. Aside of this, this trial was also designed to investigate the safety and tolerability of adding oral AL3818 dihydrochloride to standard platinum-based chemotherapy (carboplatin and paclitaxel) in patients via evaluation of dose limiting toxicity (DLT) events.

Patients in this trial were females with recurrent or advanced endometrial, ovarian, and cervical cancer.

In this presented chemo combination therapy regimen, in the first day of the 21-day cycle, standard platinum-based chemotherapeutic agents were administrated intravenously to the patients. In the embodiments of endometrial or ovarian or cervical cancer, paclitaxel (175 mg/m² infusion over 3 hours) and carboplatin (AUC 5/6 according to local standard over approximately 30 minutes) were selected as chemotherapeutic agents. In the embodiment of cervical cancer, cisplatin (at a recommended dose of 75 mg/m²) could be used instead of carboplatin. The weekly paclitaxel was used as SOC (standard of care) treatment for platinum resistant ovarian patients. 1-6 cycles of chemotherapy were applied for each individual patient.

Overall, in the presented chemo combination therapy regimen, compound AL3818 dihydrochloride capsules were administrated orally to the patients in a 14 days on and 7 days off pattern each cycle. After the intravenous (IV) chemotherapy on the Day 1, Day 2 to Day 7 was the therapy free period. From the first day of second week (Day 8), compound AL3818 (anlotinib) dihydrochloride capsules are administrated orally once daily to the patients for two weeks continuously until the end of the first 21-day cycle (Day 8 to Day 21).

From Day 22 (C2D1) (CXDY is the abbreviation for Cycle X Day Y. For example, C2D1 represents Cycle 2 Day 1), a new cycle began, paclitaxel and carboplatin (or cisplatin for cervical cancer) were administrated to the patients and Day 23 to Day 28 (C2D2-C2D7) was the therapy free period again.

The first 21-day cycle plus 7 days (C1D1-C2D8 or Day 1 to Day 28) was crucial in this regimen and it was called the primary safety evaluation period. The recommended phase 2 dose (RP2D) was obtained by evaluating according to the standard for the dose limiting toxicity (DLT) events of patients in this period of time.

This evaluation period could be extended to the end of second cycle for the patients who passed the primary safety evaluation period. From Day 29 (C2D9), compound AL3818 (anlotinib) dihydrochloride capsules were administrated orally to the patients again for two weeks until Day 42 (C2D21). After the beginning of cycle 3 (C3D1), the patients had the option to continue on chemotherapy plus AL3818 for up to 6 cycles, then AL3818 dihydrochloride in mono therapy as maintenance for up to 12 months in total. During any time of the entire study, investigations were required to see if there showed clinical benefit, no disease progression, and the tolerability after the drug administration.

In this embodiment, if any serious dose limiting toxicity (DLT) events or other serious side effects occurred, according to the serious level, the patients might require to withdraw the treatment, reduce the dose of medication or switch to other medications.

A total of nine patients were enrolled in this study and the compound AL3818 dihydrochloride dosage strength was selected from 12 mg, 10 mg and 8 mg (calculated according to the form of free base). As a result, three patients were assigned into each group, accordingly.

In the first group (12 mg AL3818 dihydrochloride), two of the patients out of three observed dose limiting toxicity (DLT) events during the primary safety evaluation period. As a result of this, one of the patients had to permanently discharge from this study and the other one selected to lower the dosage to 10 mg and continued for another two cycles. The only patient did not face dose limiting toxicity (DLT) event also selected to reduce the dosage of compound AL3818 to 10 mg on C3D1 due to intolerable chemotherapy side effects.

In the second group (10 mg AL3818 dihydrochloride), one of the patients faced dose limiting toxicity (DLT) event after receiving paclitaxel chemotherapy and 8 doses of AL3818 in the first cycle (C1D16) and the study on this patient was permanent discontinued. The other two patients in this group successfully passed the primary safety evaluation period, but shows some non-serious side effects (did not qualify DLT event standard). Due to this reason, the dosage of AL3818 was reduced to 8 mg for these two patients so that the study could go proceed.

In the third group (8 mg AL3818), no patient faced serious limiting toxicity (DLT) event. All of them passed the primary safety evaluation period smoothly and could continue being treated with this chemo combination therapy regimen.

From the results mentioned above, it was concluded that in the 21 days cycle (14 days on treatment, 7 days off), the safe daily dosage of compound AL3818 dihydrochloride was 8 mg when it was combined with the administration of chemotherapeutic agents such as paclitaxel and carboplatin (or cisplatin in the embodiment of cervical cancer). Therefore, it was decided that the recommend phase 2 dose (RP2D) for this chemo combination therapy regimen was 8 mg. The 8 mg regimen was also recommended to combine with weekly Paclitaxel SOC treatment for platinum resistant ovarian patients.

The daily dosage of 12 mg AL3818 was too high when it was administrated altogether with platinum-based chemotherapy. However, after 6 full cycles of chemo combination therapy, this dosage might become acceptable during the mono therapy period as a kind of maintenance.

A total number of nine subjects (9/9, 100%) enrolled in the study reported treatment emergent adverse events (TEAE). All subjects received at least one dose of AL3818. The most common TEAEs were nausea (9.62%), vomiting (5.77%), diarrhea (4.81%), fatigue (4.81%), alopecia (2.91%), abdominal pain (2.88%), hypertension (2.88%), dizziness (2.88%), urinary tract infection (2.88%), and weakness generalized (2.88%). Other TEAEs occurring in greater than 1% frequency included chest pain (1.92%), dehydration (1.92%), pulmonary embolism (1.92%), sore throat (1.92%), and thrombocytopenia (1.94%).

The efficacy of this chemo combination therapy was summarized in the table below. In this described study, all patients received at least one dose of AL3818 dihydrochloride orally. Aside of three patients were permanent discharged from this study due to severe adverse events (SAE), the other six patients' best response ranged from stable disease (SD) (1/6, 16.7%), partial response (PR) (4/6, 66.6%) to complete response (CR) (1/6, 16.7%).

TABLE 1
Phase 1 efficacy of AL3818 with standard platinum-based chemotherapy.

| | | Daily dosage of | |
|---|---|---|---|
| Subject No. | Cancer | AL3818 | Best response |
| 001 | Ovarian | 12 mg | NE/SAE |
| 002 | Ovarian | 12 mg | PR |
| 003 | Cervical | 12 mg | NE/SAE |
| 004 | Endometrial | 10 mg | PR |
| 006 | Ovarian | 10 mg | CR |
| 008 | Ovarian | 10 mg | NE/SAE |
| 010 | Endometrial | 8 mg | PR |
| 011 | Endometrial | 8 mg | PR |
| 012 | Ovarian | 8 mg | SD |

1. The daily dosage of compound AL3818 was the initial dosage that administrated to the patients, which might subject to a dosage reduction if serious side-effects were observed. 2. The best response represented the most favorable outcome during the entire treatment, regardless of the possible disease progression (PD) after that.

### Example 2: Representative Subject 004 in Phase 1

Subject 004 with endometrial carcinoma, who was enrolled on Jun. 17, 2016, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. The dose was reduced to be 8 mg at the third cycle and similar aforementioned treatment regimen was used in the following cycles. The maintenance mono therapy was at 8 mg once daily in 21-day cycles (14 days on treatment and 7 days off treatment). The subject was progressed to off treatment on Mar. 14, 2018 and the best response of the treatment was PR.

### Example 3: Representative Subject 010 in Phase 1

Subject 010 with endometrial carcinoma, which was enrolled on Dec. 5, 2016, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Jun. 29, 2017 (Intolerable) and the best response of the treatment was PR.

### Example 4: Representative Subject 011 in Phase 1

Subject 011 with endometrial carcinoma, which was enrolled on Dec. 6, 2016, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Sep. 13, 2017 and the best response of the treatment was PR.

### Example 5: Representative Subject 002 in Phase 1

Subject 002 with ovarian carcinoma, which was enrolled on Mar. 11, 2016, was orally administered with AL3818 dihydrochloride 12 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. The dose was reduced to be 8 mg at third cycle and similar aforementioned treatment regimen has been used in the following cycles. The maintenance mono therapy was at 12 mg once daily in 21-day cycles (14 days on treatment and 7 days off treatment). The subject was intolerable and off treatment on Sep. 6, 2016, and the best response of this treatment was PR.

### Example 6: Representative Subject 012 in Phase 1

Subject 012 with ovarian carcinoma, which was enrolled on Dec. 13, 2016, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Apr. 3, 2017, and the best response of the treatment was SD.

### Example 7: AL3818 Dihydrochloride Phase 2a Chemo Combination Initial Efficacy Evaluation Trial in Gynecologic Oncology Patients

Phase 1 was completed in Q1 2017 at one study site to determine the recommend phase 2 dose (RP2D). The RP2D was determined to be AL3818 dihydrochloride 8 mg orally once daily in 21-day cycles (14 days on treatment, 7 days off treatment) on Day 8 to Day 21 of the cycle with platinum-based chemotherapy on Day 1 of the cycle.

Phase 2a is currently ongoing at four study sites in the U.S. The first subject was enrolled in April 2017. As of December 2018, 48 subjects with recurrent or metastatic endometrial, ovarian, and cervical cancer have been enrolled. 24 subjects are still on treatment and 24 subjects have discontinued treatment. 21 endometrial cancer subjects have been enrolled, 9 subjects have discontinued treatment. 8 subjects discontinued due to disease progression, 1 subject discontinued due to toxicity. 12 subjects remain on treatment.

AL3818-US-002 Phase 2a Subject Disposition

| | |
|---|---|
| Endometrial | 21 |
| Ovarian | 23 |
| Cervical | 4 |

Combining Phase 1 and Phase 2a subjects, 24 subjects with endometrial cancer have been enrolled in study AL3818-US-002. 19 subjects have had evaluable responses as of December 2018. There were eight subjects enrolled in Phase 2a of the study as first-line treatment. Objective response rate (ORR) was 62.5% (5/8) and disease control rate (DCR) was 75% (6/8). There were five partial responses (PR) as best responses.

Eleven subjects enrolled in Phase 1 and Phase 2a of the study as second- and further-line treatment. Objective response rate (ORR) was 54.5% (6/11) and disease control rate (DCR) was 82% (9/11). There was one complete response (CR) and five partial responses (PR) as best responses.

The most common TEAEs were abdominal pain (4.20% of all TEAEs), alopecia (1.3%), anaemia (1.8%), arthralgia (1.6%), asthenia (2.2%), back pain (1.5%), constipation (2.7%), decreased appetite (2.7%), diarrhea (6.3%), dyspnea (1%), epistaxis (3%), fatigue (4.5%), flatulence (1.5%), headache (3.2%), hypertension (3.3%), hypokalemia (1.3%), hypomagnesaemia (1%), insomnia (1.3%), nausea (4.7%), neuropathy peripheral (2%), neutropenia (2%), neutrophil count decreased (1%), pain in extremity (1.7%), pyrexia (1.2%), thrombocytopenia (1.7%), urinary tract infection (1.3%), vomiting (2.8%), and weight decreased (1%).

All efficacy results were listed in Table 2 and Table 3

TABLE 2
Phase 2a efficacy of AL3818 with standard
platinum-based chemotherapy on
endometrial cancer patients (Dec 2018).

| | AL3818 + CB/PAC | First | Trial | Best | |
|---|---|---|---|---|---|
| No. | PFS(mo) | Line | Lines | Response | Phase |
| 015 | 7 | 1st | | PR | 2a |
| 016 | 20 (on study) | 1st | | PR | 2a |
| 017 | 15 | | 2nd | SD | 2a |
| 026 | 8 | 1st | | SD | 2a |
| 029 | 2 | | 3rd | PD | 2a |
| 037 | 9 | | 5th | SD | 2a |
| 038 | 8.5 | | 2nd | CR | 2a |
| 046 | 6 | 1st | | PR | 2a |
| 047 | 9 (on study) | | 2nd | PR | 2a |
| 049 | 6 (on study) | | 3rd | PR | 2a |
| 050 | 3 (good CA125) | | 2nd | PD | 2a |
| 051 | 3 | 1st | | PD | 2a |
| 054 | 5 (on study) | | 2nd | SD | 2a |
| 058 | 2 (only C3 on drug) | 1st | | PD | 2a |
| 059 | 3 (on study) | 1st | | PR | 2a |
| 061 | 3 (on study) | 1st | | PR | 2a |

TABLE 3
Phase 2a efficacy of AL3818 with standard platinum-based
chemotherapy on ovarian cancer patients. (Dec 2018)

| Subject | Best Response | prior |
|---|---|---|
| No | (% reduction) | lines |
| 014 | SD | 1 |
| 018 | NE (was not treated) | |
| 023 | NE (was not treated) | |
| 024 | SD | 1 |
| 030 | PD | 1 |
| 032(PtR) | PR | 1 |
| 033 | NE/Intolerability | |
| 034 | Investigator Decision | 3 |
| 036(PtR) | PR | 1 |
| 038 | PR | 1 |
| 043(PtR) | SD | 6 |
| 044 | SD | 3 |
| 045 | PR | 5 |
| 053(PtR) | SD | 3 |
| 055(PtR) | NE/Intolerability | |

PtR: Platinum Resistant

### Example 8: Representative Subject 015 in Phase 2a

Subject 015 with endometrial carcinoma, which was enrolled on Apr. 9, 2017, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Nov. 3, 2017, and the best response of this treatment was PR.

### Example 9: Representative Subject 017 in Phase 2a

Subject 017 with endometrial carcinoma, which was enrolled on Apr. 24, 2017, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Aug. 1, 2018, and the best response of this treatment was SD.

### Example 10: Representative Subject 026 in Phase 2a

Subject 026 with endometrial carcinoma, which was enrolled on Jul. 12, 2017, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Mar. 19, 2018, and the best response of this treatment was SD.

### Example 11: Representative Subject 037 in Phase 2a

Subject 037 with endometrial carcinoma, which was enrolled on Oct. 19, 2017, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Jul. 16, 2018, and the best response of this treatment was SD.

### Example 12: Representative Subject 038 in Phase 2a

Subject 038 with endometrial carcinoma, which was enrolled on Oct. 29, 2017, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Jul. 9, 2018, and the best response of the treatment was CR.

### Example 13: Representative Subject 046 in Phase 2a

Subject 046 with endometrial carcinoma, which was enrolled on Mar. 16, 2018, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-day cycles (14 days on treatment and 7 days off treatment). The subject was progressed to off treatment on Sep. 9, 2018, and the best response of the treatment was PR.

### Example 14: Representative Subject 024 in Phase 2a

Subject 024 with ovarian carcinoma, which was enrolled on Aug. 14, 2017, was orally administered with AL3818 dihydrochloride 8 mg once daily in 21-day cycles (14 days on treatment from Day 8 to Day 21 and 7 days off treatment from Day 1 to Day 7 of cycle one) in combination with standard paclitaxel and carboplatin chemotherapy in Day 1. Similar aforementioned treatment regimen was used in the following cycles. The maintenance mono therapy was at 8 mg once daily in 21-Day cycles (14 days on treatment and 7 days off treatment). The subject was progressed to off treatment on Feb. 27, 2018, and the best response of this treatment was SD.

### Example 15: AL3818 Combination Therapy with Immunotherapy Agents Sequentially Use

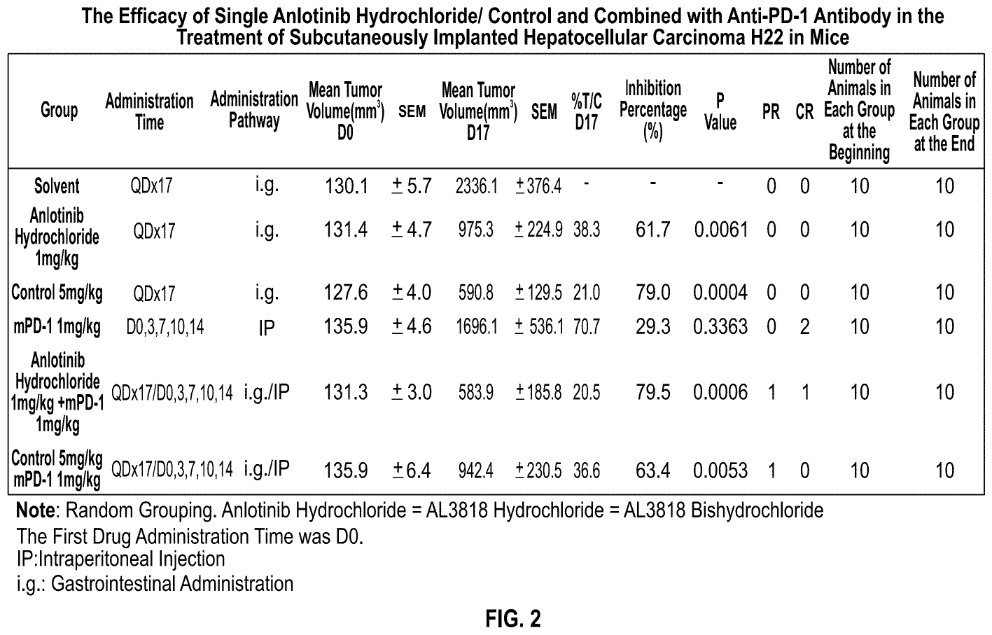

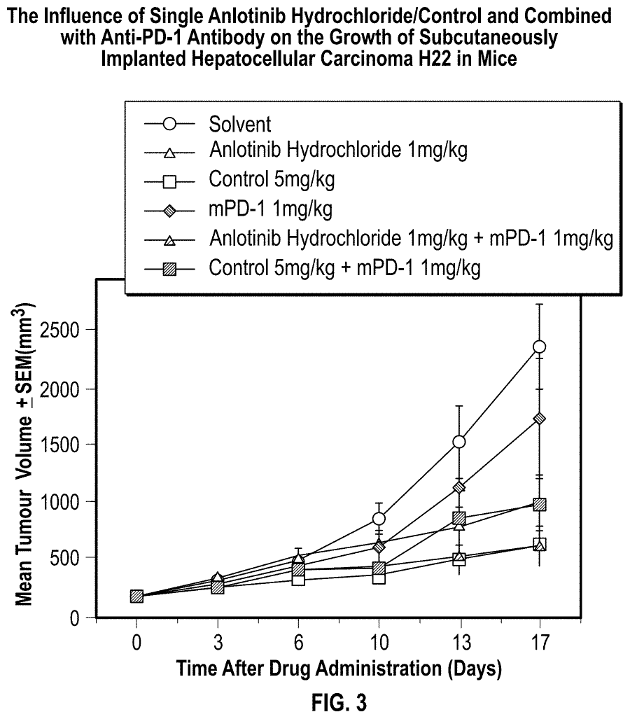

Based on the inventor's research experience using AL3818 free base, its HCl salts (mono- or bis-), its bis-maleic acid salt and its succinic salt, the following in clinical combining effects are expected, which in combining with immunotherapy agents, selected from PD-1 or PD-L1, SLAM7, oncolytic virus therapy, bispecific T cell engagers (BiTE) and chimeric antigen receptor (CAR) T cell therapy based agents, such as nivolumab, pembrolizumab, ipilimumab, blinatumomab, elotuzumab, daratumumab, cemiplimab, avelumab, durvalumab, atezolizumab, toripalimab, sintilimab, camrelizumab, tislelizumab, AK105, KN035, CS1001, talimogene laherparepvec. but not limited, in treating solid tumors, selected from lung, renal, colorectal, gastric, melanoma, head/neck, thyroid, pancreatic, liver, prostate, bladder, brain, sarcoma, breast, ovarian, cervical and endometrial cancers; and blood cancers, selected from ALL, CLL, AML, CML and Multiple Myeloma. The similar sequential treatment regimen which waiting 0-7 days, preferably 0 day or 7 days, to administrate AL3818 or its pharmaceutical acceptable salts after administration of above immunotherapy agents as a combination therapy method could generate synergistic and combined anti-tumor activities. A tumor xenograft model combing with murine PD-1 antibody has been conducted to show combination results in FIG. 2 and FIG. 3. Usually, 50% to >100% regression in vivo tumor inhibition activities are expected on various solid tumor cell lines, such as lung (such as but not limited: NSCLC and SCLC), renal, colorectal, gastric, melanoma, head/neck, thyroid, pancreatic, liver, prostate, bladder, brain, sarcoma, breast, ovarian and cervical cancers; and blood cancers, such as ALL, CLL, AML, CML and Multiple Myeloma.

### Example 16: AL3818 Combination Therapy with Immunotherapy Agent Nivolumab

AL3818 and Nivolumab administrations are both started on Day 1. Nivolumab is administrated via infusion on Day 1 and Day 15 for twice per 28 days, 21 days as one cycle will still be applied for the combination therapy. Nivolumab can be administrated via infusion on Day 1 once per 28 days, 21 days as one cycle will still be applied for the combination therapy. AL3818 is administrated orally from Day 1 to Day 14 and off from Day 15 to Day 21 for 21 days as one cycle. Nivolumab is administrated via infusion on Day 1 and Day 15 for twice per 28 days starting dose at 240 mg twice per 28 days. Nivolumab can be optionally administrated via infusion on Day 1 once per 28 days at 480 mg. AL3818 is administrated orally at Day 1-Day 14 at starting dose of 12 mg.

### Example 17: AL3818 Combination Therapy with Nivolumab Phase I Trial

Treatments of subject 001 (liposarcoma), 002 (synovial sarcoma), 003 (angiosarcoma), 004 (leiomyosarcoma), 005 (undifferentiated pleomorphic sarcoma) and 006 (leiomyosarcoma) at 10 mg oral dosing Day 1-14 combing with Nivolumab at Day 1 and Day 15 for twice per 28 days starting dose at 240 mg infusion have been completed with one DLT (subject 002). Dose escalation to 12 mg combination therapies with Nivolumab with cervical cancer subject and other sarcoma subjects are ongoing.

What is claimed is:

1. A regimen method to treat cancer in a subject in need thereof comprising:
a standard platinum-based chemotherapeutic agent and another chemotherapeutic agent; or each individually; or an immunotherapeutic agent; in combing with AL3818 or its pharmaceutically acceptable salts in repeated cycles with the option of administration of maintenance mono therapy of AL3818 or its pharmaceutically acceptable salts.

2. The method of claim 1, the administration of chemotherapeutic agents or an immunotherapeutic agent with AL3818 or its pharmaceutically acceptable salts is repeated periodically in subsequent order.

3. The method of claim 1, the administration of chemotherapeutic agents or an immunotherapeutic agent with AL3818 or its pharmaceutically acceptable salts is repeated periodically on a 21-day cycle for 1-10 cycles until patient intolerability or progression disease.

4. The method of claim 1, the chemotherapeutic agents for standard platinum-based chemotherapy and another chemotherapy agent or an immunotherapeutic agent are administrated once on the first day (Day 1) of the 21-day cycle followed by another 21-day cycle for 6 cycles.

5. The method of claim 1, the chemotherapeutic agents for standard platinum-based chemotherapy and another chemotherapy agent or an immunotherapeutic agent are administrated once on the first day (Day 1) of the 21-day cycle followed by another 21-day cycle continuously until patient intolerability or progression disease.

6. The method of claim 1, AL3818 or its pharmaceutically acceptable salts is administrated in a pattern of 14 days on treatment and 7 days off treatment each cycle.

7. The method of claim 1, AL3818 or its pharmaceutically acceptable salts is administrated in a pattern of 14 days of 21-day cycle on treatment daily from Day 1 to Day 14 and 7 days off treatment from Day 15 to Day 21 of each cycle.

8. The method of claim 1, AL3818 or its pharmaceutically acceptable salts is preferably administrated in a pattern of 14 days of 21-day cycle on treatment daily from Day 8 to Day 21 and 7 days off treatment from Day 1 to Day 7 of each cycle

9. The method of claim 1, the chemotherapeutic agents for standard platinum-based chemotherapy is selected from carboplatin, cisplatin and another chemotherapeutic agent is paclitaxel.

10. The method of claim 1, the chemotherapeutic agents used in the treatment is selected from carboplatin and paclitaxel together or weekly paclitaxel.

11. The method of claim 1, the chemotherapeutic agent is selected from gemcitabine, gemcitabine/docetaxel, paclitaxel, pegylated liposomal doxorubicin (PLD) and topotecan.

12. The method of claim 1, a standard platinum-based chemotherapeutic agent and another chemotherapeutic agent or an immunotherapeutic agent; in combing with AL3818 or its pharmaceutically acceptable salts in 1-6 cycles followed by maintenance mono therapy of AL3818 or its pharmaceutically acceptable salts until patient intolerability or progression disease.

13. The method of claim 1, the daily dosage of compound AL3818 or its pharmaceutically acceptable salts is 8 mg in combination period and 8 mg in maintenance mono therapy period.

14. The method of claim 1, the daily dosage of compound AL3818 or its pharmaceutically acceptable salts is 8 mg in combination period and 10 mg or 12 mg in maintenance mono therapy period.

15. The method of claim 1, the daily dosage of compound AL3818 or its pharmaceutically acceptable salts is at 14 mg or 16 mg.

16. The method of claim 1, the cancer includes, lung, renal, colorectal, gastric, melanoma, head/neck, thyroid, pancreatic, liver, prostate, bladder, brain, sarcoma, breast, ovarian and cervical cancers; and blood cancers, ALL, CLL, AML, CML and Multiple Myeloma.

17. The method of claim 1, the cancer includes recurrent or advanced endometrial, ovarian, and cervical cancer.

18. The method of claim 1, the immunotherapy agents are PD-1 or PD-L1 antibodies and these PD-1 or PD-L1 antibodies are selected from nivolumab, pembrolizumab, ipilimumab, blinatumomab, elotuzumab, daratumumab, cemiplimab, avelumab, durvalumab, atezolizumab, toripalimab, sintilimab, camrelizumab, tislelizumab, AK105, KN035, CS1001, talimogene laherparepvec.
